(19) United States
(12) Patent Application Publication
(10) Pub. No.: US 2010/0043804 A1
(43) Pub. Date: Feb. 25, 2010

(54) Oral Cavity Manipulator

(76) Inventor: John Razmovski, Victoria (AU)

Correspondence Address: LETHAM LAW FIRM, LLC, 914 N. TUCANA LANE, GILBERT, AZ, 85234, US

(21) Appl. No.: 11/721,811

(22) PCT Filed: Dec. 15, 2005
(86) PCT No.: PCT/AU05/01898

§ 371 Date: Oct. 15, 2009

(30) Foreign Application Priority Data
Dec. 15, 2004 (AU) 2004907149

(51) Int. Cl. A61F 5/44

(52) U.S. Cl. 128/848

(57) ABSTRACT

The present application is directed to an improved oral cavity manipulator (1) including at least one pair of oppositely disposed teeth engaging elements (2, 3) adapted to be located on or over the teeth on opposite sides of the oral cavity; and a tongue depression means (4) interconnecting rear portions of the at least one pair of teeth engaging elements (2, 3), wherein the tongue depression means (4) urges against a surface portion of the user's tongue, close to the throat, when the manipulator is operatively positioned so that the tongue is displaced downwardly and forwardly to substantially avoid obstruction of airways.

### FIELD OF INVENTION

The present invention relates to an oral cavity manipulator that in use restrains movement of a user's tongue to substantially prevent obstruction of a user's airways. The present invention particularly relates to a device for substantially preventing snoring and Obstructive Sleep Apnoea (OSA). In particular the present invention relates to a device for insertion within the oral cavity of a snorer and an OSA sufferer (or non-sufferer/user) which effectively maintains the sufferer's tongue in a substantially constant position irrespective of movement and/or shifting position of the sufferer's head/body.

### BACKGROUND OF THE INVENTION

Obstructive Sleep Apnoea is a disease which affects up to about 7% of the Australian population. Symptoms of the disease OSA manifest themselves in the form of fatigue, tiredness, poor concentration, mood changes and in some instances impotence has been reported. If left untreated and in some instances undiagnosed the disease can be very debilitating and even fatal. Indeed it is known that sufferer's of OSA are more likely to have motor vehicle accidents and are at greater risk of heart attack and stroke.

OSA is understood to be caused by blocking, of the airway as a result of muscle relaxation of the throat during sleep. An episode of apnoca may expose sufferers to full or partial obstruction, and hence breathing can stop for durations of between 10 seconds to over 2 minutes.

During an episode a sufferer is prompted to awake when the brain registers an adverse change in blood gases, usually a drop in oxygen concentration. Waking from an apnoea episode is usually followed by a short period of rapid hyperventilation to correct the blood gas concentrations. This process produces a series of snorts and gasps before sleep resumes and the cycle repeated. A sufferer with severe OSA may have as many as 500 episodes per night with sleep occurring at intervals of time no longer than the longest period of apnoea (say 60 sec).

The act of waking during an apnoea episode is indicative of the symptoms of fatigue, and tiredness during the day. Hence higher frequency of episodic apnoea gives rise to increased severity of symptoms. As stated above a characteristic of OSA is the substantial gasping for air during a waking period. This results from the presence of an occluded airway requiring a need to expend a substantial inspiratory effort to overcome occlusion and to initiate the waking phase. In comparison, a non-sufferer or mild sufferer needs only to expend slight inspiratory effort if airways become occluded, hence they do not tend to arouse from sleep.

Some risk factors associated with OSA include weight gain; alcohol consumption; intake of sedative(s); and sleeping position. These factors are treatable by simple therapeutic measures. For example, recent studies have shown that, while obesity is a risk factor, the tendency to have OSA is more closely correlated with neck circumference. It has been postulated that excess weight in the neck reduces the ability of the pharyngeal muscles to maintain a lumen (opening), during sleep when ‘tonic postural input is withdrawn’. In this case weight loss may be sufficient to address sleep problems associated with OSA.

Other risk factors mentioned such as alcohol can be treated by modifying consumption. In numerous cases it has been found that some people are disposed to OSA symptoms if lying supine. This can be helped by measures which effectively prevent sleeping in such a position.

OSA sufferers can be treated in a number of ways. For example people exhibiting an increased episodic severity may require positive pressure therapy (PPT) or wearing of an oral appliance. The most common form of PPT is referred to as ‘continuous positive airway pressure’ (CPAP). In general CPAP is administered through a nasal or face mask strapped to a person's head. The mask is connected by a hose to an air compressor and the compressor sends air under pressure through the hose into the mask. The positive air pressure blows open the pharynx and prevents the airway from collapsing. Such a device however suffers from the disadvantage(s) that it is not aesthetically pleasing; is relatively expensive; is uncomfortable; can initiate nose bleeds and renders the nose and sinus cavity feeling dry. Alternative devices such as tongue-restraining devices relying on suction have been used in which a suction cup is gripped with the teeth and sucks the tongue forward to open the airways behind the tongue.

Oral appliances have been used for the treatment of OSA including mandibular advance devices which involve pulling the mandible (lower jaw) forward relative to the maxilla (upper jaw). The practical effect of advancing the lower jaw is that the sufferer's tongue follows the jaw displacement forward so that it is less able to cause obstruction of the airway when a sufferer is sleeping. Mandibular devices are generally less expensive than CPAP devices and therefore offer an economic alternative. Mandibular advancement devices to date however do not prevent or operate to stop a sufferer's tongue from rolling as the sufferer alternates his or her sleeping positional. It is known that in a sleeping condition the muscles of the throat and tongue relax. While a mandibular advancement device is able to position the lower jaw forward of the upper jaw, the tongue may still interfere with the airway opening particularly when a sufferer moves the position of his/her head. On movement of a person's head position from one side to the other, momentum exerted by head movement is sufficient to move the tongue from its initial resting position. In many instances Tolling of the tongue may result in airway obstruction thus lowering the efficiency of an oral mandibular advance device.

One object of the invention is therefore to provide an alternative oral cavity appliance that substantially addresses one or more of the problems of the prior art.

### SUMMARY OF THE INVENTION

In order to address one or more of the problem(s) of the prior art devices the present invention is directed to an improved oral cavity manipulator including:

at least one pair of oppositely disposed teeth engaging elements adapted to be located on or over the teeth on opposite sides of the oral cavity; and
a tongue depression means interconnecting rear portions of the at least one pair of teeth engaging elements, wherein the tongue depression means urges against a surface portion of the user's tongue, close to the throat, when the appliance is operatively positioned.

One advantage of the present invention is that in a fitted condition the depression means of the oral cavity appliance is able to exert a directional force on a base surface portion of the tongue near the throat thus urging the tongue towards a forward position. At the same time a user's tongue is confined between the depression means and the teeth engaging elements. Hence rolling movement of a user's tongue that would otherwise occur in a relaxed state is substantially obviated to allow unobstructed passage of air even when a user moves from side to side during a sleeping cycle.

As the user's tongue is urged forward by the depression means the tongue makes positive contact with side portions of the pair of teeth engaging elements. The action of the tongue against side portions of the pair of teeth engaging elements helps to retain the oral appliance within the oral cavity. The oral cavity manipulator of the invention can therefore be used by persons without teeth. The oral appliance can include teeth or gum mounting portions extending forwardly of the at least one pair of teeth engaging elements. To further assist in maintaining the oral appliance within a user's oral cavity, the mounting portions can include a flange to substantially prevent rearward displacement of the oral appliance. The flange can be external or internal of the mounting portions. Throughout the specification the term ‘teeth engaging elements’ includes gum engaging elements and lower jaw(s).

The oral cavity manipulator can include a tongue supporting means extending from or adjacent to the at least one pair of teeth engaging elements, the tongue support being disposed relative to the tongue depression means to provide support for the tongue.

In use when the oral cavity manipulator is fitted within the oral cavity of a user, the user's tongue is trapped between the depression means, the tongue supporting means and the at least one pair of teeth engaging elements so that the tongue is substantially prevented from uncontrolled rolling and concomitant airway obstruction when the user changes resting position during sleep.

The tongue depression means can extend in a direction substantially in the same plane as the teeth engaging elements. The positioning of the depression means relative to the teeth engaging elements in such a construction allows the depression means to positively urge against a base surface portion of the user's tongue, close to the throat, when the oral cavity appliance is operatively fitted/positioned. The tongue depression means can alternatively extend either below or above the plane of the teeth engaging elements. The depression means can be resiliently biased or can be a rigid structure.

The depression means can be substantially u- or v-shaped. The depression means can be shaped such that the oral appliance exhibits symmetry about an axis located between the oppositely disposed teeth engaging means. The depression means can include a curved shallow ‘m’ shape in cross-section. In this construction of the depression means, the depression means has a shallow trough interposed between adjacent curved peaks such that in a fitted condition the trough is able to exert a positive downward force against a surface of a user's tongue near the base portion of the tongue adjacent the throat. In a fitted condition the user's tongue is urged forward of and away from the epiglottis or obstructive position, hence lessens the ability of the tongue to create an airway obstruction. The user's tongue is thus able to be urged forward as well as downwards away from an obstructive position. When the trough portion of the depression means is urged against the base of the tongue, in an operative condition, the tongue can adopts a similarly curved shape such that a valley can be formed in the central part of the tongue to create further distance away from an obstructive position.

The teeth engaging means can include a structure having a wall and a roof member, the roof member being joined to the wall at its upper edge such that the roof and the wall of each of the teeth engaging members provides a general shape which is adapted to make engaging contact with part or all surfaces of at least some or all of a user's maxilla and/or mandibular dental arch.

The teeth engaging means of the oral cavity manipulator can include a gripping means to help retain/maintain engagement contact between the teeth engaging means and the user's teeth. The gripping means can include a skirt comprising a plurality of legs integral with and extending from a peripheral edge of the roof member(s). Each leg forming the skirt can be straight or curved and ends in a foot or enlarged end. The foot or enlarged end can make contact with a surface of a tooth, opposite to the wall, to help retain the teeth engaging members in engaged relation to a user's teeth.

The tongue supporting means can include a flange member extending adjacent to or from a lower edge of the wall of each of the at least one pair of teeth engaging elements to form oppositely disposed shoulders on which at least a part of the tongue of a user can be seated when the oral cavity appliance is in a fitted condition. The flange member can include a base support for supporting an underneath base portion of a wearer's tongue closer to the throat end of the wearer. The base support can include a pair of cradle members extending equally and oppositely from the shoulder portions of the flange member. The pair of cradle members can be disposed at one end of the oppositely disposed shoulder portions closest to the tongue depression means. The oral cavity manipulator can be adapted to fit on or in relation to the maxilla and/or mandibular dental arches. The oral cavity appliance can also include absorption means positioned relative to the at least one pair of teeth engaging means to relieve pressure if mandibular and maxilla teeth adopt are clenched. In one embodiment of this aspect the teeth engaging means can be made from or include an absorbing medium.

In a fitted condition the tongue of a user is seated on the shoulder portions of each flange member and is supported by the cradle means. The depression means consequently acts against a base portion of the user's tongue and the tongue is urged forward. In this condition the tongue substantially prevented from uncontrolled rolling or collapsing because it is trapped within boundaries defined by (a) the walls of the oppositely disposed pair of teeth engaging means; (b) shoulder portions of the support means; and (c) the tongue depression means. Retention of the tongue in this manner substantially maintains open airways by minimizing the propensity of the tongue to move in a rearward and/or rolling action during sleep. Indeed the oral cavity manipulator of the invention serves to retain the tongue of a user in a substantially constant position relative to the position of the user's head position. This provides a significant advantage in terms of lessening the potential for airway obstruction. The pair of cradle means can further aid restriction of tongue movement while providing optimum support for the user's tongue during sleep.

In a second aspect of the invention there is disclosed an oral cavity manipulator for use in substantially preventing incidences of OSA or snoring, the appliance including:

at least one pair of oppositely disposed teeth engaging elements adapted to be located on or over the teeth on opposite sides of the oral cavity;
a tongue depression means interconnecting rear portions of the at least one pair of teeth engaging elements, wherein the tongue depression means urges against a surface portion of the sufferer's tongue, close to the throat, when the appliance is operatively positioned; and
a tongue supporting means extending from or adjacent to the at least one pair of teeth engaging elements, the tongue support being disposed relative to the tongue depression means to provide support for the tongue.

In a third aspect of the present invention there is described an oral cavity manipulator including:

a mandibular oral appliance having a pair of oppositely disposed mandibular teeth engaging elements adapted to be located on or over teeth on opposite sides of a sufferer's teeth on opposite sides of the sufferer's lower dental arch;
a maxilla oral appliance having a pair of oppositely disposed maxilla teeth engaging elements adapted to be located on or over teeth on opposite sides of the sufferer's teeth on opposite sides of the sufferer's upper dental arch wherein the mandibular and maxilla oral appliances are hingedly interconnected by a connecting means to allow movement of the mandibular appliance relative to the maxilla appliance;
a tongue depression means for exerting a forward directional force on a rearward surface portion of the tongue of a sufferer when in a fitted condition, the tongue depression means interconnecting rear portions of the pair of oppositely disposed mandibular teeth engaging elements, wherein the tongue depression means extends behind the mandibular teeth engaging elements such that the depression means is urged against a base portion of the sufferer's tongue, close to the throat, when the appliance is operatively positioned;
a body member interconnecting the pair of maxilla teeth engaging elements; and
a tongue support means extending from or adjacent to the mandibular teeth engaging elements for supporting the tongue, the tongue support and the depression means being relatively disposed so that the sufferer's tongue is substantially restrained from collapsing rearwards and/or sideways by the depression means in a supported condition between the pair of maxilla teeth engaging means.

The body member, that interconnects the pair of maxilla (upper) teeth engaging members, can be hollow having a forward opening to allow ingress of air into the hollow body. The hollow body can include at least one outlet rearwardly of the body for egress of air following transfer of air from the forward opening through the body upon a breathing intake cycle. One advantage of this arrangement is that the sufferer's oral cavity is less prone to dryness.

The body can include a humidifying chamber in which air entering the body is moistened and heated by heating elements mounted within the chamber. Moistened, warm air thus exits a rear portion of the body and enters the trachea during a normal inspiratory cycle. One advantage of the humidifying chamber is that the vapor entering the trachea is heated and thus helps to avoid intake of cold air. A further advantage is that the chamber can aid breathing of a user by generating a vapor from a menthol or eucalyptus medicament or the like.

The humidifying chamber can include at least one heating coil which heats incoming air to substantially minimize intake of cold air by the user. The chamber can also include a filter(s) for collection of dust or other particulate matter from normal air intake.

The connection means can include a flexible hinge system extending from the at least one outlet of the body member to the depression means. The depression means can include two rib type members extending from a common neck portion in a wishbone type structure such that each rib member is joined at its end to the teeth engaging elements comprising the first pair of oppositely disposed teeth engaging elements. The two rib members can be tubular or at least partially tubular ending in an opening at the neck portion which is seated adjacent the throat of a wearer in a fitted condition.

The flexible hinge system can include a pair of tubular members extending from a pair of spaced apart outlets positioned rearwardly of the body member. In this arrangement inhaled air is drawn into the hollow body via the forward opening and exited through the rearward outlets. Air subsequently enters the partially tubular rib members, which communicate with the tubular hinge members, and exits the opening in the neck portion.

The body can further include an array of baffles suspended from an internal surface of the hollow body which acts as a filter to absorb any dust particles or other air borne matter. The connecting members can include a concertina structure to accommodate for any type of relative angular movement of upper or lower jaw.

The teeth engaging members include a wall which is joined at its upper edge to a roof member extending in a direction substantially transverse to the wall. The roof member and the wall of each of the teeth engaging members provide a general shape which is adapted to make engaging contact with the contours of a person's lower and upper dental arch. Each of the teeth engaging means can include gripping means that extend from the peripheral edge of the corresponding roof member.

The tongue supporting means can include a flange member extending adjacent to or from a lower edge of the wall of each of the at least one pair of teeth engaging elements to form oppositely disposed shoulders on which at least a part of the tongue of a wearer can be seated when the oral appliance is in a fitted condition. The flange member can include a base support for supporting an underneath base portion of a wearer's tongue closer to the throat end of the wearer. The base support can include a pair of cradle members extending equally and oppositely from the shoulder portions of the flange member. The pair of cradle members can be disposed at one end of the oppositely disposed shoulder portions closest to the tongue depression means.

In a fourth aspect of the invention there is described a method of substantially preventing or at least minimising incidences of OSA and/or snoring, the method including:

providing a device which can be inserted within the oral cavity of a user/sufferer, the device having a mandibular teeth engaging structure and a tongue depression means attached to the teeth engaging structure, wherein the tongue depression means extends rearwardly of the teeth engaging structure so that in a fitted condition the tongue depression means acts against a base portion of the users/sufferers tongue;
wherein when the device is fitted within the oral cavity, the users/sufferers tongue is deflected forwardly to maintain a substantially unobstructed airway.

In a fifth aspect of the invention there is described a method of substantially preventing or at least minimising incidences of OSA and/or snoring, the method including:

providing an oral cavity manipulator for fitting within a sufferer's oral cavity, the oral cavity appliance having at least one pair of oppositely disposed teeth engaging elements adapted to be located on or over the teeth on opposite sides of the oral cavity; and
a tongue depression means interconnecting rear portions of the at least one pair of teeth engaging elements
wherein the method includes fitting the oral cavity appliance within the oral cavity of a sufferer so that the tongue depression means urges against a base surface portion of the user's tongue, close to the throat, when the appliance is operatively positioned and the sufferer's tongue is restrained between the teeth engaging elements and the tongue depression means to substantially minimise movement of the sufferer's tongue.

In a further related aspect of the present invention there is disclosed an oral cavity manipulator including:

u-shaped maxilla and mandibular appliances, the said appliances being hingedly attached and having a u-shaped cross-section for receiving teeth of a wearer and wherein the appliances include hollow portions along their length;
an air inlet connected to a front portion of the maxilla and mandibular appliances, the air inlet communicating with the hollow portions of the appliances to allow air transfer through the appliances;
a tongue depression means connected to and located aft of the appliances wherein the tongue depression means includes a platform ending in a moveable depression element for urging against a base portion of a wearer's tongue, and wherein the tongue depression means includes hollow portions ending in air outlet ports such that continuous passage of air flows from the air inlet to air outlets in an inspiratory cycle;
wherein in an operating condition the tongue depression element is angularly positionable by the wearer relative to the platform to exert a force on a base portion of the wearer's tongue and thereby cause the tongue to displace downwardly and forwardly within the oral cavity.

In this related aspect of the present invention, the oral cavity manipulator is adjustable to alter the angle of the tongue depression element relative to the maxilla and mandibular appliances. A wearer can therefore make adjustments to suit the force required to displace the tongue away from an airway obstructing position.

The platform can include a series of parallel spaced ribs to allow angular adjustment of the tongue depression element with respect to the maxilla and mandibular appliances.

The air inlet can be shaped to allow attachment of hose connection to an external air supply. The tongue depression means can be a separate attachable structure, attachable to rear portions of the appliances. In one embodiment the tongue depression means can include strengthening elements which extend into the hollow portions of the maxilla and mandibular appliances.

The tongue depression means can also include a shaped tongue depression element. In particular the tongue depression element can be u- or v-shaped and include a central opening. The applicant has suggested, although not wishing to be bound by theory, that the central opening may when wetted cause a partial vacuum and help retain the wearer's tongue in a holding/displaced condition.

The oral cavity manipulator can further include tongue support members.

### BRIEF DESCRIPTION OF THE DRAWINGS

FIG. 1 is a perspective view of an oral cavity manipulator in accordance with one embodiment of the present invention from a partial side elevation;

FIG. 2 is a rear perspective view of the oral cavity manipulator of FIG. 1 from a partial side elevation;

FIG. 3 is a cross-sectional view of the oral cavity manipulator shown in FIGS. 1 & 2 shown in a fitted condition;

FIGS. 4a, b, c, are each a plan view (top elevation) of the oral cavity manipulator of FIGS. 1 & 2 in a fitted condition and FIG. 4d is a cross-sectional representation of the oral cavity manipulator in-situ;

FIG. 5 is a cross-sectional representation of the oral cavity manipulator in accordance with FIGS. 1 and 2 in a user's oral cavity;

FIG. 6 is a perspective view of an oral cavity manipulator in accordance with a second embodiment of the present invention shown in partial side elevation;

FIG. 7 is a rear perspective view of the oral cavity manipulator in accordance with FIG. 6 shown in partial side elevation; and

FIG. 8 is a cross-sectional view of the oral cavity manipulator in accordance with FIGS. 6 and 7 fitted within a user's oral cavity.

FIGS. 9a, 9b, 9c, 9d, and 9e represent front, underneath, side, top, and rear views respectively of an alternative embodiment in accordance with the present invention.

FIG. 10 illustrates a side view of a further related embodiment according to the present invention.

### DETAILED DESCRIPTION OF THE PREFERRED EMBODIMENTS WITH REFERENCE TO THE ACCOMPANYING DRAWINGS

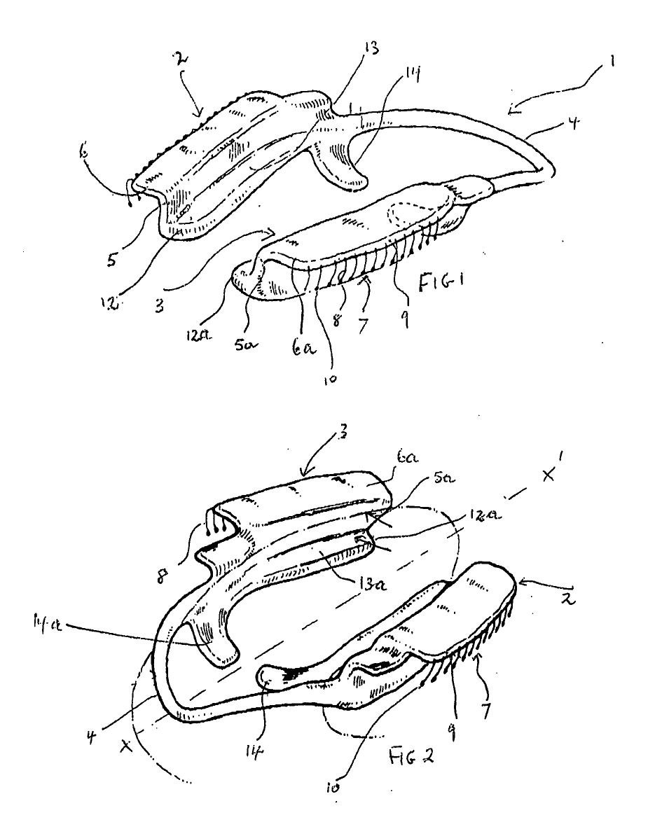

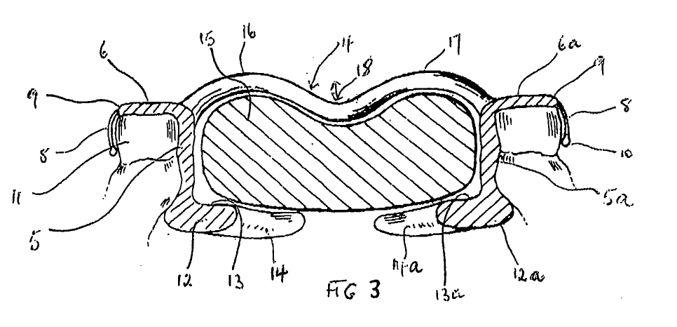

The present invention is directed to an oral cavity manipulator which restricts movement of a user's tongue when in a fitted condition. The oral cavity manipulator can also be useful in substantially preventing snoring and/or OSA. Referring to FIGS. 1 to 5 there is shown an oral cavity manipulator 1 in accordance with one embodiment of the present invention which is adapted to maintain open airways and substantially prevent airway obstruction by limiting movement of a wearer's tongue with change in sleeping position of the user/sufferer while asleep. The oral cavity manipulator illustrated in FIGS. 1 to 5 has a generally ‘U’- or ‘V’-shaped configuration so that the appliance can be located on or over teeth on opposite sides of the oral cavity of the lower (mandibular) dental arch of a user or OSA sufferer. When fitted within a user's/sufferer's oral cavity the manipulator restrains movement of the user's/sufferer's tongue and substantially maintains airways in an unobstructed condition.

The oral cavity manipulator 1 includes a pair of oppositely disposed teeth engaging elements/appliances 2 and 3 adapted to be located on or over the teeth on opposite sides of the mandibular dental arches (best seen in FIGS. 3 to 5). Each teeth engaging element/appliance in the pair of oppositely disposed teeth engaging elements are interconnected by a tongue depression means 4 attached at rear portions of each teeth engaging elements in the pair. The teeth engaging elements 2 and 3 include a wall 5 and 5a which is upstanding having an upper edge and a lower edge. Joined at the upper edge of the wall there is shown a roof member 6 and 6a extending outwardly therefrom in a direction substantially transverse to the wall. The roof member and the wall of each of the teeth engaging members in the pair provide a general shape which is adapted to make engaging contact with the contours or tooth surfaces of a user's lower (mandibular) dental arch.

To help retain the teeth engaging members 2 and 3 in contact with a user's teeth, the teeth engaging members include a skirt 7 extending downwardly from a peripheral edge 9 of the roof members 6 and 6a distal from the top edge of the wall. The skirt 7 comprises a plurality of spaced apart legs 8 extending outwardly and downwardly from the peripheral edge 9. Each of the leg members ends in an enlarged end portion 10 which contacts a surface of a tooth adjacent the gum line 500. As shown best in FIG. 3 the teeth engaging means encase a substantial surface area of a tooth 11 to help maintain the oral cavity appliance in a constant position in use.

The oral cavity manipulator 1 further includes a flange 12 and 12a extending inwardly from a lower edge portion of the walls 5 and 5a. The flanges 12 and 12a form a longitudinal shoulder 13 and 13a ending in cradle members 14 and 14a respectively. In use (see FIG. 3) the tongue 15 of a user/sufferer rests on the shoulder portions 13 and 13a and is additionally supported by the each of the cradle members 14 and 14a further back towards the base of the tongue. The cradle members extend inwardly from rear portions of the flanges 12 and 12a in a slightly curved configuration.

The oral cavity manipulator also includes a tongue depression means 4. As best shown in FIGS. 1, 2 and 4 the depression means has a general wishbone configuration which interconnects the pair of teeth engaging elements at a rear portion of the teeth engaging elements 2 and 3. In use the tongue depression means urges against a surface or base portion of the user's/sufferer's tongue, close to the throat causing the tongue to displace both downwardly and forwardly. In this embodiment the mandibular oral appliance is symmetric about an axis x-x′. The manipulator can be made of material which is pliable, for example when subject to heating conditions, to enable fitting within an individuals oral cavity structure.

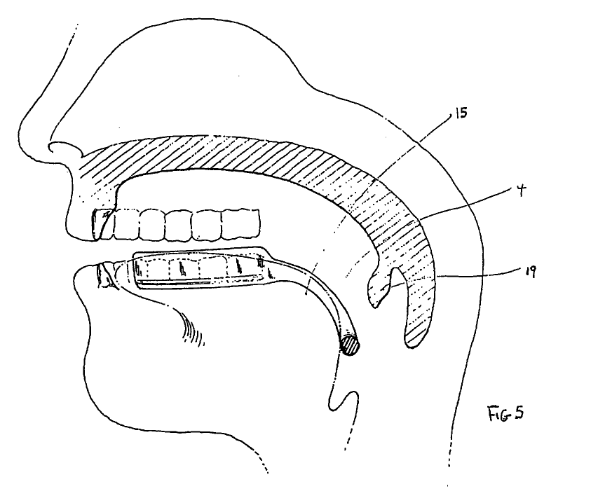

Referring to FIG. 3 the tongue depression means 4 has an undulating cross section shaped in a shallow ‘m’ configuration which is defined by adjacent curved peaks 16 and 17 separated by a central trough 18. In use the depression means exerts a downward pressure on a wearer's tongue at or adjacent the base of the throat and urges the tongue to adopt a corresponding ‘valley’ shape so as to lessen the likelihood of airway obstruction. As shown in FIG. 5 the depression means 4 urges against a rear or base portion of the user's tongue 15 away from the user's epiglottis 19 to maintain clear passage for flow of air. In FIG. 3 the base portion of the tongue is shown to adapt a central valley configuration corresponding to the trough part of the depression means to displace the tongue away from an occluded position.

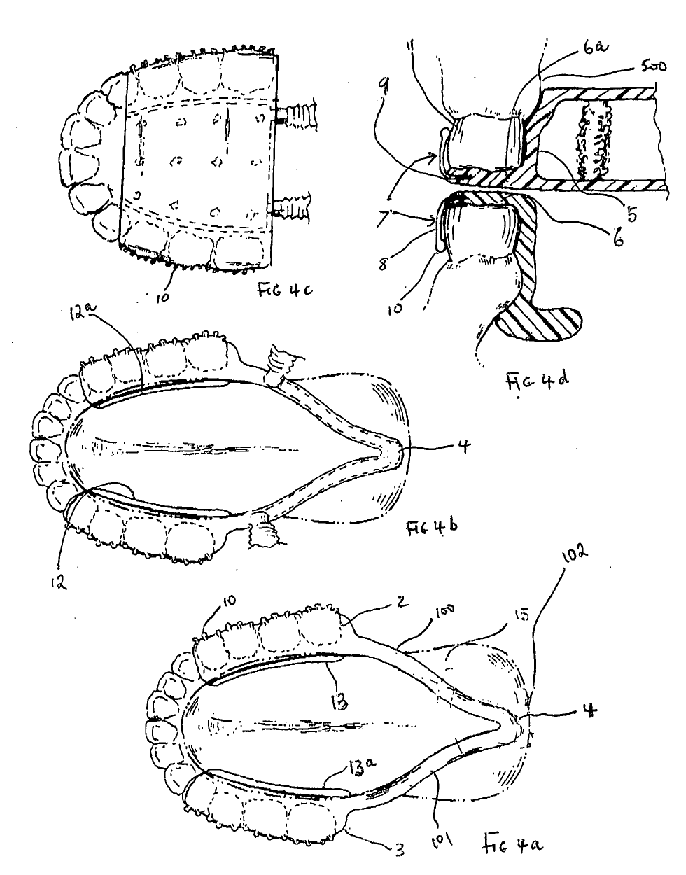

Referring to FIG. 4 there is shown a representation of the oral cavity manipulator in accordance with FIG. 1 in a fitted condition in relation to a user's/sufferer's tongue and lower dental arch. As can be seen the pair of teeth engaging elements 2 and 3 engage four teeth on opposite sides of the lower dental arch. The oral cavity manipulator allows the user's/sufferer's tongue to be positioned comfortably between the depression means 4 and the shoulders 13 and 13a. The oral manipulator is constructed so that the depression means extends behind and in-line or slightly above the plane of the pair of teeth engaging elements. In a fitted/operational condition the depression means urges the tongue forward and away from an obstruction position relative to the throat. Once in this position the depression means helps to capture the user's/sufferer's tongue between the teeth engaging elements, while the tongue is supported in a resting condition by the shoulder supports and cradle means. In this way a user's/sufferer's tongue is substantially prevented from uncontrolled rolling motion and collapsing which can otherwise become obstructive as a wearer's sleeping position is changed. FIG. 4 also illustrates a ‘v’-shape or wishbone configuration having arm or rib type members 100 and 101 connected to and extending rearwardly from rear portions of the respective teeth engaging elements in the pair of oppositely disposed mandibular teeth engaging elements 2 and 3. The arm or rib type members 100 and 101 converge to a common end portion 102 which in a fitted condition rests against a central base portion of the user's tongue close to the user's throat. The end portion 102 corresponds with trough 18 in FIG. 3. The arm or rib members also curve upwardly from the end portion 102 corresponding to arched peak portions 16 and 17 in FIG. 3. The end portion 102 can be elongated to extend further towards or into a user's throat (not shown). The elongation can also include a convex bottom portion for resting on a surface of the tongue, close to the throat, so that the volume of air flow during inspiratory effort is substantially independent of a user's sleeping position.

Turning to FIGS. 6 to 8 there is disclosed an oral cavity manipulator 20 having an upper (maxilla) oral appliance 21 and a lower (mandibular) oral appliance 22 which are hingedly interconnected to allow movement of one appliance relative to the other. The maxilla appliance includes a pair of oppositely disposed teeth engaging elements 23 and 23a located on or over at least some of the teeth on opposite sides of the maxilla dental arch. Similarly the mandibular appliance includes a pair of oppositely disposed teeth engaging elements 24 and 24a located on or over at least some of the teeth on opposite sides of the mandibular dental arch. The maxilla dental arch oppositely disposed pair of teeth engaging elements 23 and 23a are interconnected by a centrally located planar/rectangular hollow body 25. The pair of oppositely disposed mandible teeth engaging elements is interconnected at rear portions thereof by a ‘v’-shaped tongue depression means 26.

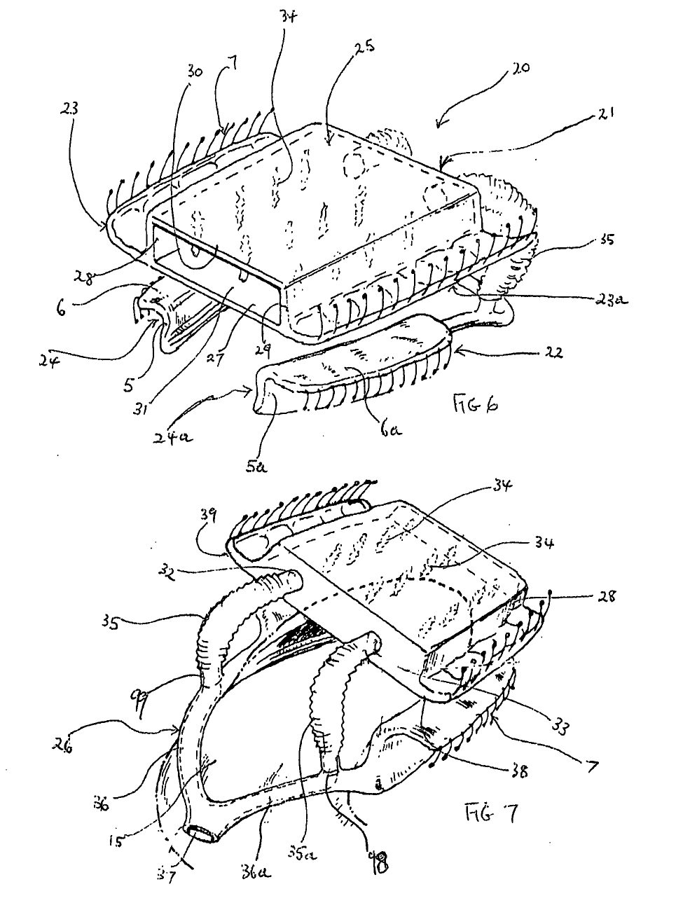

As best seen in FIGS. 6 and 7 the hollow body 25 has a floor 27 from which there extends dependent side walls 28 and 29 and a ceiling section 30. The hollow body has an opening 31 at a front portion of the maxilla appliance through which air is drawn by a user/sufferer into the hollow interior. The hollow body also includes exit openings 32 and 33 at a rear portion to allow egress of air. In use air enters the hollow portion through the opening during inspiratory action and air entering the body is able to exit the body via the exit openings at the rear of the hollow body. The body further includes an array of baffles 34 suspended from the ceiling which act as filtering elements to help absorb any dust particles or other air borne matter.

In one embodiment of the present invention the hollow body acts as a humidifying chamber. In this embodiment the baffles 34 represent an array of miniature heating coils and filter elements. The heating coils initiate condensation of water particles suspended in air and subsequently apply heat so that the moistened air is warmed before exiting rear openings in the body.

The maxilla and mandibular appliances are interconnected by means of hollow connecting members 35 and 35a. The hollow connecting members are shown in FIG. 7 connecting the exit openings 32 and 33 to the depression means. The hollow connecting members connecting the lower and upper dental arch appliances allow further passage of air exiting from the hollow body. In FIGS. 6 and 7 the hollow connecting members comprise flexible tubing in a concertina structure to accommodate a range of jaw positions and angles corresponding to relative movement of upper and lower jaw. The flexible tubing is adapted to fit over inlet ports 98 and 99 shown on the depression means although the tubing can be made as an integral component. The tubing as shown is configured in an arc or an ‘L’ shape to fit within the contour of the maxilla part of the oral cavity.

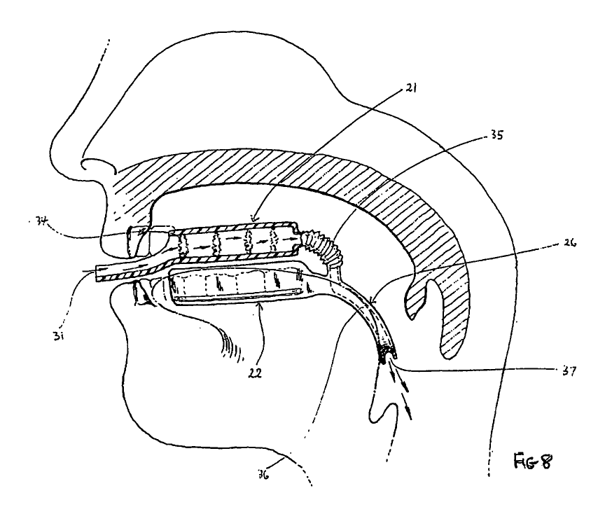

Referring to FIGS. 7 and 8 it can be seen that the depression means includes a hollow section 36 and 36a which communicates with the hollow connecting members, ending in an exit opening 37 for egress of inspiratory air. Following the cross sectional representation in FIGS. 7 and 8, air enters the opening of the hollow body during inspiratory effort and passes over or through the baffles on their way to the connecting members. Air then flows through the connecting members and enters the hollow section of the depression member, thereafter exiting the depression member via exit the opening 37.

The teeth engaging members in each pair of teeth engaging members of the upper and lower dental arch oral appliances include a wall 28, 29 and 5, 5a respectively, which are joined to respective roof members 38, 39 and 6, 6a extending outwardly in a direction substantially transverse to the each wall. The combined roof and wall structures of each pair of teeth engaging members provide a general shape which is adapted to make engaging contact with the contours of a person's lower and upper dental arch. As shown each of the teeth engaging means include gripping means 7 that extend from the peripheral edge of the corresponding roof member. Referring to FIGS. 7 and 8 there is shown an oral cavity manipulator in accordance with FIGS. 6 and 7 in a fitted condition. As shown a sufferer's tongue 15 is positioned below the depression means and is seated on shoulder portions 13 and 13a of the mandibular appliance. As the sufferer's tongue is urged forward by the action of the tongue depression means the tongue also is displaced sideways and engages the walls 5 and 5a of the pair of mandibular teeth engaging elements.

In a fitted condition the force of the tongue against opposite side walls of the mandibular teeth engaging elements helps to brace the teeth engaging elements against the teeth and/or gums of the user/sufferer hence substantially preventing the oral appliance from inadvertent removal. In such a condition the tongue is trapped between boundaries defined by opposite side walls 5 and 5a and is restrained from collapsing rearwardly by constant force exerted on a base portion of the tongue by the depression means. The tongue of a user or sufferer is thus confined and prevented from substantial movement both rearwardly and sideway collapse thus restraining any urge the tongue would otherwise have of such movement in a relaxed state and maintaining an obstructed position for continual open passage of air.

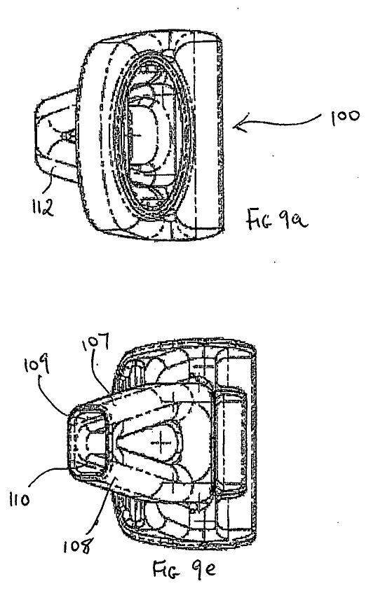

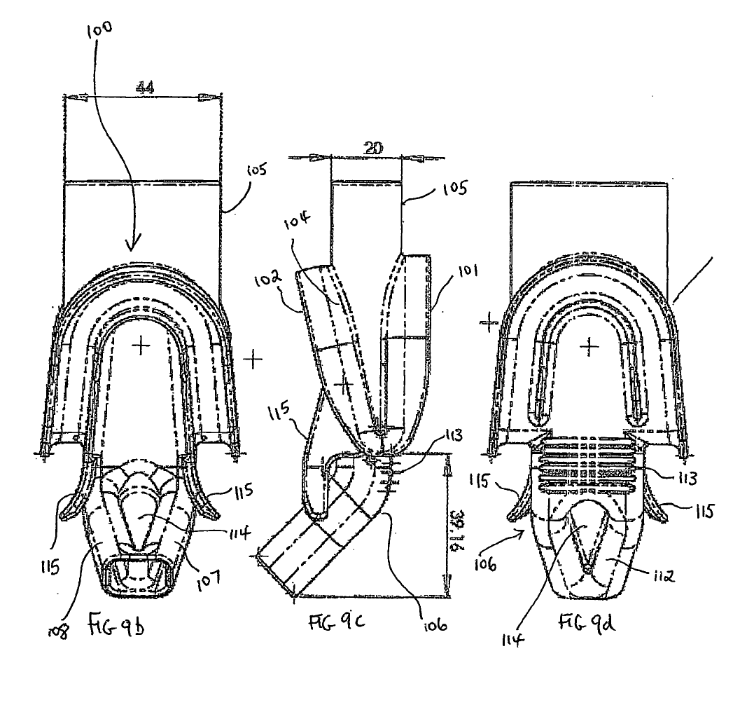

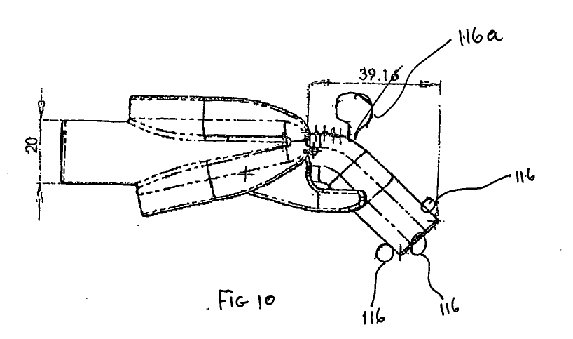

Referring to FIGS. 9a to 9e there is shown a further embodiment of the present invention illustrating an oral cavity manipulator 100 including a maxilla 101 and mandibular 102 appliances (best seen in FIG. 9c) hingedly connected. The maxilla and mandibular appliances are u-shaped and include a u-shaped cross-sectional recess for receiving a wearer's teeth. In one aspect the maxilla and mandibular appliances are fabricated from a fluid plastic material which conforms to the shape of the wearer's teeth and helps retain the manipulator within the oral cavity while in use.

The maxilla and mandibular appliances include hollow portions 103, 104 along their length, which communicate with an air inlet 105 to allow transfer of air from the air inlet.

The manipulator has a tongue depression means 106, which in this embodiment is shown as a unitary structure being integrally formed with the maxilla and mandibular appliances. It should be noted however that the tongue depression means can alternatively comprise a separate component adapted to mate with the maxilla and mandibular appliances. And in a related aspect the tongue depression means can include a strengthening member (not shown), which extend within the hollow portions of the maxilla and mandibular appliances to provide a strengthening element.

In this embodiment, the tongue depression means includes hollow portions 107 and 108, which provide a continuous airflow pathway from the air inlet at the front of the manipulator to the rear of the manipulator where air exits outlet ports 109 and 110 close to the base portion of a wearers tongue. The air inlet is also shaped to accommodate a hose connection so that an external air supply can be administered.

The tongue depression means 106 includes a platform 111 extending from the rear of the maxilla and mandibular appliances and ends with a v- or u-shaped extension member 112. The platform incorporates a plurality of parallel spaced ribs 113, which allow angular positioning of the extension member relative to the platform. As can be seen the ribs are able to flex to allow suitable positioning of the extension member to exert a displacement force against the base of the tongue of a wearer. The plurality of ribs are sufficiently resilient to retain the extension member in a desired position and allow further adjustment or return to a planar position relative to the platform.

The extension member 112 includes an opening 114, which when subjected to wetting by saliva tends to form a vacuum and thus helps to urge and retain the tongue of a wearer in a desired position. As can be seen in FIG. 9c, the oral cavity manipulator further includes a tongue support 115, which supports a portion of the wearer's tongue in an operating condition.

In a further embodiment (see FIG. 10), the oral cavity manipulator has incorporated therein a number of diaphragms 116, which expand on intake of air. In the embodiment illustrated, a number of diaphragms are shown in various stages of expansion. It is envisaged by the applicant that one diaphragm 116a can be used to obstruct the upper airways to substantially minimise escape of inspiratory air from the nasal passages. Other diaphragms can be used to supplement the extension member to advance the tongue forward in the oral cavity and maintain flow of air.

1.-41. (canceled)

42. An oral cavity manipulator comprising:
a mandibular teeth engaging element adapted to be located on or over teeth of a sufferer's lower dental arch, and a maxilla teeth engaging element adapted to be located on or over teeth of a sufferer's upper dental arch, wherein the mandibular and maxilla elements are hingedly interconnected by a connecting means to allow movement of the mandibular element relative to the maxilla element;
a tongue depression means connected to and located aft of the mandibular and maxilla teeth engaging elements for exerting a forward directional force on a rearward surface portion of the tongue of a sufferer when in a fitted condition, wherein the tongue depression means extends behind and beneath the plane of the mandibular and maxilla teeth engaging elements so as to urge against a base portion of the sufferer's tongue, close to the throat when the teeth engaging elements are operatively positioned;
a body member interconnecting the maxilla and mandibular teeth engaging elements wherein the body member is hollow having a forward opening to allow ingress of air into the hollow body and at least one outlet rearwardly of the body for egress of air following transfer of air from the forward opening through the body upon a breathing intake cycle; and
a tongue support means extending from or adjacent to the mandibular teeth engaging element for supporting the tongue, the tongue support and the depression means being relatively disposed so that a sufferer's tongue is substantially restrained from collapsing rearwards and/or sideways by the depression means in a supported condition between the maxilla and mandibular teeth engaging elements.

43. The oral cavity manipulator of claim 42 wherein the tongue depression means extends in a direction substantially in the same plane as the teeth engaging elements wherein in an operating/fitted condition the depression means urges against a base surface portion of the user's tongue, close to the throat, to help maintain open airways.

44. The oral cavity manipulator of claim 43 wherein the tongue depression means comprises two rib-type members extending from a common neck portion in a wishbone type structure such that each rib member is joined at its end to the maxilla and mandibular teeth engaging elements.

45. The oral cavity manipulator of claim 44 wherein the two rib members are tubular or at least partially tubular ending in an opening at the neck portion which is seated adjacent the throat of a wearer in a fitted condition.

46. The oral cavity manipulator of claim 42 wherein the tongue depression means comprises a platform disposed between the maxilla and mandibular teeth engaging elements and a tongue contacting element dependent therefrom, wherein the platform comprises a series of parallel spaced ribs to allow angular adjustment of the tongue contacting element with respect to the maxilla and mandibular elements.

47. The oral cavity manipulator of claim 42 wherein the connecting means comprises a flexible hinge system extending from the at least one outlet of the body member to the depression means, and wherein the flexible hinge system comprises a pair of tubular members extending from a pair of spaced apart outlets positioned rearwardly of the body member, wherein air is drawn into the hollow body via the forward opening and exited through the rearward outlets.

48. The oral cavity manipulator of claim 42 wherein the connecting means comprises a concertina structure to accommodate for any type of relative angular movement of upper or lower jaw.

49. The oral cavity manipulator of claim 42 wherein the maxilla and mandibular teeth engaging elements comprise gripping means that extend from the peripheral edge of the corresponding roof member.

50. The oral cavity manipulator of claim 42 wherein the tongue supporting means comprises a flange member having a base support for supporting an underneath base portion of a wearer's tongue close to the throat end of the wearer wherein the flange member extends adjacent to or from a lower edge of the wall of each of the at least one pair of teeth engaging elements to form oppositely disposed shoulders on which at least a part of the tongue of a user is able to be seated when the oral cavity appliance is in a fitted condition.

51. The oral cavity manipulator of claim 50 wherein the base support comprises a pair of cradle members disposed at one end of the oppositely disposed shoulder portions closest to the tongue depression means, the cradle members extending equally and oppositely from the shoulder portions of the flange member to support the tongue.

52. The oral cavity manipulator of claim 42 wherein the teeth engaging elements comprise a gripping means to help retain/maintain engagement contact between the teeth engaging means and the user's teeth, the gripping means comprises a skirt comprising a plurality of legs integral with and extending from a peripheral edge of the roof member(s) wherein each leg forming the skirt is straight or curved and ends in a foot or enlarged end, wherein the foot or enlarged end contacts a surface of a tooth, opposite to the wall, to help retain the teeth engaging members in engaged relation to a user's teeth.

53. The oral cavity manipulator of claim 42 wherein the body member comprises a humidifying chamber in which air entering the body is moistened and heated by heating elements mounted within the chamber.

54. The oral cavity manipulator of claim 53 wherein the humidifying chamber comprises at least one heating coil which heats incoming air to substantially minimize intake of cold air by the user.

55. The oral cavity manipulator of claim 54 wherein the chamber further comprises at least one filter for collection of dust or other particulate matter from normal air intake.

56. The oral cavity manipulator of claim 42 wherein the body member further comprises an array of baffles suspended from an internal surface of the hollow body which acts as a filter to absorb any dust particles or other air borne matter.

57. An oral cavity manipulator comprising:
u-shaped maxilla and mandibular teeth engaging elements, the said elements being hingedly attached and having a u-shaped cross-section for receiving teeth of a wearer and wherein the elements include hollow portions along their length;
an air inlet connected to a front portion of the maxilla and mandibular elements, the air inlet communicating with the hollow portions of the elements to allow air transfer through the elements;
a body member interconnecting the pair of maxilla teeth engaging elements wherein the body member is hollow having a forward opening to allow ingress of air into the hollow body and at least one outlet rearwardly of the body for egress of air following transfer of air from the forward opening through the body upon a breathing intake cycle, and wherein the body includes a humidifying chamber in which air entering the body is moistened and heated by heating elements mounted within the chamber;
a tongue depression means connected to and located aft of the elements wherein the tongue depression means includes a platform and a tongue contacting element, the platform having a series of parallel spaced ribs for allowing angular adjustment of the tongue contacting element relative to the platform for applying an effective downward and forward displacement force on the tongue of a user for urging against a base portion of a wearer's tongue, and wherein the tongue depression means includes hollow portions ending in air outlet ports such that continuous passage of air flows from the air inlet to air outlets in an inspiratory cycle;
a tongue support means extending from or adjacent to the mandibular teeth engaging element for supporting the tongue, the tongue support and the tongue depression means being relatively disposed so that a sufferer's tongue is substantially restrained from collapsing rearwards and/or sideways by the depression means in a supported condition between the maxilla and mandibular teeth engaging elements; and
wherein in an operating condition the tongue depression element is angularly positionable by the wearer relative to the platform to exert a force on a base portion of the wearer's tongue and thereby cause the tongue to displace downwardly and forwardly within the oral cavity.
